(19) United States
(12) Patent Application Publication
(10) Pub. No.: US 2011/0004257 A1
(43) Pub. Date: Jan. 6, 2011

(54) Implant Structure

(75) Inventors: Ming-Chang Wu, Taoyuan County (TW); Jia-Jyun Hong, Taoyuan County (TW); Chi-Bin Wu, Taoyuan County (TW)

Correspondence Address: STITES & HARBISON, PLLC, Suite 900, 1199 N. Fairfax Street, Alexandria, VA, 22314, US

(73) Assignee: AccuMIS Inc., Zhonghe City (TW)

(21) Appl. No.: 12/535,706

(22) Filed: Aug. 5, 2009

(30) Foreign Application Priority Data
Jul. 3, 2009 (TW) 098122636

(51) Int. Cl. A61B 17/86

(52) U.S. Cl. 606/309

(57) ABSTRACT

An implant structure is provided. The implant structure includes an implant body and a screw portion. The screw portion is coupled to a second surface of the implant body such that the implant structure can be implanted into an injured bone via the screw portion. Thus, the implant body partially replaces and thereby repairs the injured bone. Threads of the screw portion have at least a barb unit for preventing the implant structure from rotating in the loosening direction of the screw portion. The at least a barb unit also prevents the implant structure from loosening and displacement so that the service life of the implant structure can be extended.

### BACKGROUND OF THE INVENTION

1. Technical Field

The present invention relates to implant structures and, more particularly, to an implant structure for repairing an injured bone.

2. Description of Related Art

Owing to advancement of modern medicine, life expectancy is increasing, though at the cost of an increasing number of patients at advanced age. The elderly are likely to suffer from degenerative arthritis. At its early stage, degenerative arthritis is treated with medication for relief of symptoms. However, patients with degenerative arthritis will have to receive artificial implant replacement surgery if degenerative arthritis lames them or deforms their joints.

The purpose of artificial implant replacement surgery is to excise an injured joint and replace the excised joint with an artificial implant so as to restore mobility of the joint. At present, artificial implants are used in treating injured shoulder joints, elbow joints, wrist joints, interphalangeal joints, hip joints, knee joints, ankle joints, etc.

To completely assume the function of an injured joint, an artificial implant has to be made of a sophisticatedly designed material that meets the requirements for high rigidity, wear resistance, ease of shaping, etc. Most importantly, an artificial implant should have a service life long enough to allow a patient to use the artificial implant for decades without changing the artificial implant.

During the artificial implant replacement operation, an artificial implant must be firmly fixed in position to a bone so that the artificial implant will not get loose after frequent and lengthy use. Nonetheless, as joints of the human body are in use and subjected to forces in all directions every day, it is not uncommon that an artificial implant in a patient's body loosens or even displaces. As a result, not only will the patient's daily life be inconvenienced, but also the adjacent disease-free bones are endangered.

Accordingly, there is still room for improvement in addressing the loosening problem facing artificial implants so as to enable artificial implants to be firmly fixed in position to bones without getting loose after frequent and long-term use.

### BRIEF SUMMARY OF THE INVENTION

The present invention provides an implant structure characterized in that the implant structure includes a screw portion having a barb unit for preventing the implant structure from rotating in a loosening direction after the implant structure is implanted into a bone, thereby effectively preventing the implant structure from loosening.

The present invention provides an implant structure characterized in that the implant structure, once implanted into a bone, is prevented from loosening and thereby is prevented from displacement.

To achieve the above and other effects, the present invention provides an implant structure including: an implant body having a first surface and a second surface adjoining a periphery of the first surface; and a screw portion coupled to the second surface and provided with threads having at least a barb unit.

Implementation of the present invention at least involves the following inventive steps:

1. With the barb unit, the implant structure is prevented from loosening.

2. The barb unit that prevents the implant structure from loosening further prevents displacement of the implant structure.

### BRIEF DESCRIPTION OF THE SEVERAL VIEWS OF THE DRAWINGS

The invention as well as a preferred mode of use, further objectives, and advantages thereof will be best understood by referring to the following detailed description of an illustrative embodiment in conjunction with the accompanying drawings, wherein:

FIG. 1 is a perspective view of an embodiment of an implant structure according to the present invention;

FIG. 2 is a partial perspective view of an embodiment of a screw portion according to the present invention;

FIG. 3 is a partial perspective view of another embodiment of the screw portion according to the present invention; and

FIG. 4 is a partial perspective view of yet another embodiment of the screw portion according to the present invention.

### DETAILED DESCRIPTION OF THE INVENTION

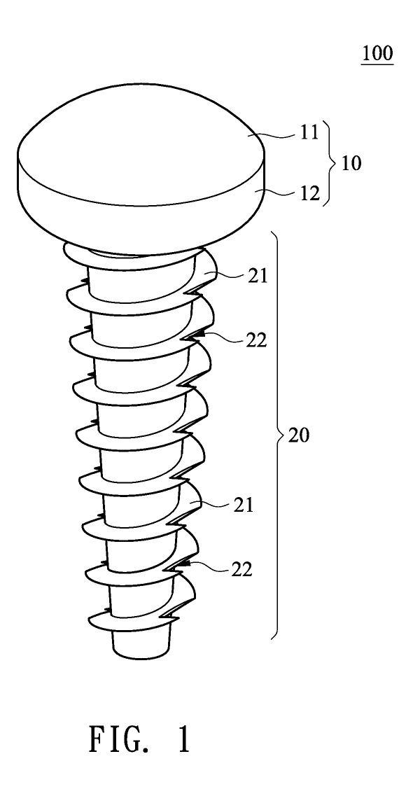

Referring to FIG. 1, in an embodiment of the present invention, an implant structure 100 includes an implant body 10 and a screw portion 20.

As shown in FIG. 1, the implant body 10 has a first surface 11 and a second surface 12. The second surface 12 adjoins a periphery of the first surface 11. The first surface 11 and the second surface 12 are made according to the profile of an injured portion of a bone. Thus, the injured portion of the bone can be completely replaced by the implant body 10, and the functions of the bone can be restored by the implant replacement. The implant body 10 is made of an alloy or a metal.

With reference to FIG. 1, the screw portion 20 is coupled to the second surface 12 of the implant body 10. Hence, the implant structure 100 can be implanted into an injured bone via the screw portion 20. The screw portion 20 is provided with threads 21 having at least a barb unit 22. The screw portion 20 is made of an alloy or a metal.

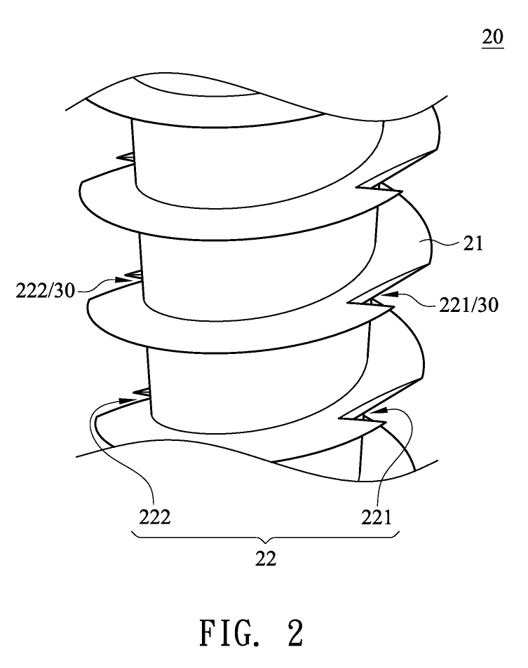

Referring to FIG. 2, the at least a barb unit 22 of the screw portion 20 includes a first barb unit 221 and a second barb unit 222. As shown in the drawing, the first barb unit 221 and the second barb unit 222 are provided opposite to each other on the edge of the same thread 21. In other words, the first barb unit 221 and the second barb unit 222 are provided on the same thread 21 and form an included angle of 180 degrees.

The first barb unit 221 and the second barb unit 222 of the screw portion 20 are each a hook-shaped cut 30. The hook-shaped cut 30 is oriented along the thread 21 in a way that allows tightening of the screw portion 20 but does not allow loosening of the screw portion 20. Hence, the hook-shaped cut 30 functions as a barb when the implant structure 100 is rotated in the loosening direction of the screw portion 20.

For instance, when screwed in the tightening direction of the screw portion 20, the implant structure 100 can be positioned deep in a bone. However, when the implant structure 100 is turned in the loosening direction of the screw portion 20, the hook-shaped cuts 30 that are provided in the implant structure 100 and are so oriented as to not allow loosening of the screw portion 20 further engage with the bone. Thus, the implant structure 100 is locked even more firmly in the bone and is effectively prevented from rotating further in the loosening direction of the screw portion 20. Consequently, the implant body 10 is prevented from loosening and displacement.

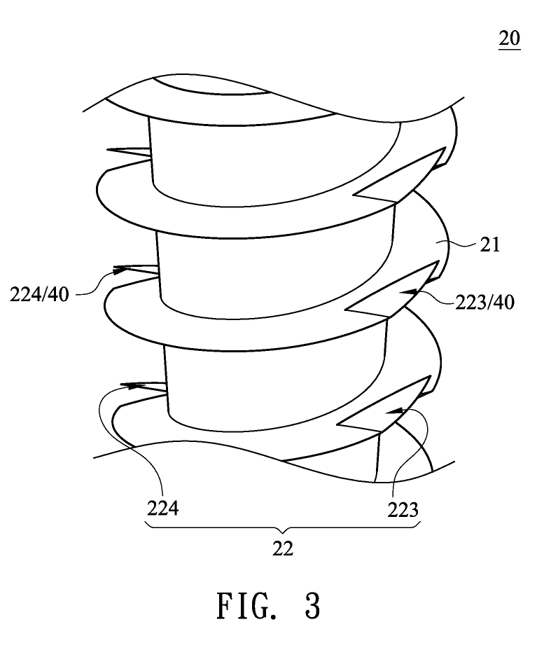

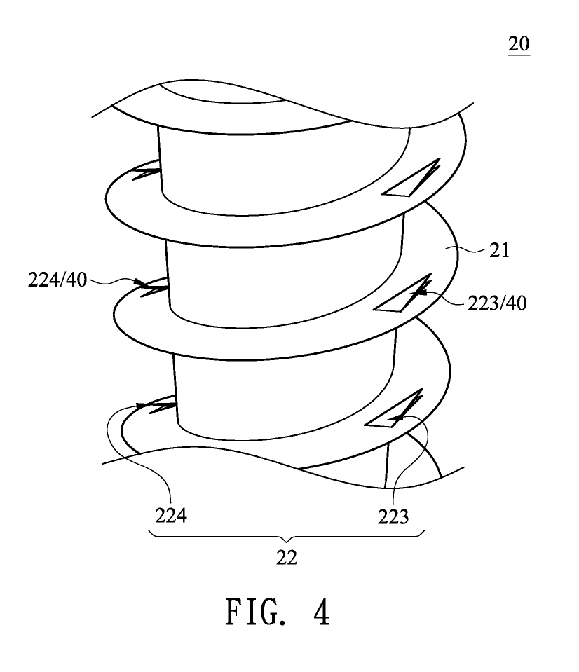

Referring to FIG. 3 and FIG. 4, the at least a barb unit 22 of the screw portion 20 includes a third barb unit 223 and a fourth barb unit 224. As shown in the drawing, the third barb unit 223 and the fourth barb unit 224 are provided opposite to each other on a surface of the same thread 21. In other words, the third barb unit 223 and the fourth barb unit 224 are provided on a surface of the same thread 21 and form an included angle of 180 degrees. Alternatively, the at least a barb unit 22 includes a wing 40.

As shown in FIG. 3, the wings 40 are formed on the edge of a surface of the same thread 21. Alternatively, referring to FIG. 4, the wings 40 are formed on a surface of the same thread 21. The projecting tip of each of the wings 40 points in a direction against loosening of the screw portion 20. When the implant structure 100 is rotated in the tightening direction of the screw portion 20 and locked in the bone, the wings 40 are also rotated in the tightening direction of the screw portion 20 and locked in the bone. However, as soon as the implant structure 100 is rotated in the loosening direction of the screw portion 20, the wings 40 are forced open and engage with the bone, thereby preventing the loosening and displacement of the implant structure 100.

The foregoing embodiments are illustrative of the characteristics of the present invention so as to enable a person skilled in the art to understand the disclosed subject matter and implement the present invention accordingly. The embodiments, however, are not intended to restrict the scope of the present invention. Hence, all equivalent modifications and variations made in the foregoing embodiments without departing from the spirit and principle of the present invention should fall within the scope of the appended claims.

What is claimed is:

1. An implant structure, comprising:
an implant body having a first surface and a second surface adjoining a periphery of the first surface; and
a screw portion coupled to the second surface and provided with threads having at least a barb unit.

2. The implant structure of claim 1, wherein the implant body is made of an alloy or a metal.

3. The implant structure of claim 1, wherein the screw portion is made of an alloy or a metal.

4. The implant structure of claim 1, wherein the at least a barb unit comprises a first barb unit and a second barb unit provided opposite to each other on an edge of a same said thread.

5. The implant structure of claim 1, wherein the at least a barb unit comprises a third barb unit and a fourth barb unit provided opposite to each other on a surface of a same said thread.

6. The implant structure of claim 1, wherein the at least a barb unit comprises a third barb unit and a fourth barb unit provided opposite to each other on an edge of a surface of a same said thread.

7. The implant structure of claim 1, wherein the at least a barb unit comprises a hook-shaped cut.

8. The implant structure of claim 1, wherein the at least a barb unit comprises a wing.
